(19) United States
(12) Patent Application Publication
(10) Pub. No.: US 2010/0281605 A1
(43) Pub. Date: Nov. 11, 2010

(54) EAR MUFFS

(76) Inventor: Jorge Miguel PEREIRA, Palmerston (AU)

Correspondence Address: LADAS & PARRY LLP, 224 SOUTH MICHIGAN AVENUE, SUITE 1600, CHICAGO, IL, 60604, US

(21) Appl. No.: 12/773,147

(22) Filed: May 4, 2010

(30) Foreign Application Priority Data
May 6, 2009 (AU) 2009201810

(51) Int. Cl. A42B 1/24; A61F 9/00; A42B 1/06

(52) U.S. Cl. 2/422; 2/11; 2/209

(57) ABSTRACT

Ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on a carrier portion adapted to be supported on a wearer's head.

### FIELD OF THE INVENTION

The present invention relates to ear muffs and particularly to ear muffs that are configurable to multiple different uses.

### BACKGROUND ART

Ear muffs are known. Conventional ear muffs include a unitary headband portion having a crown portion which rests across the crown of a user's head and a pair of arm members which extend downwardly. Each arm member is provided with a noise suppression portion mounted thereto adjacent a lower end such that the noise suppression portion can be positioned to overlie a wearer's ears.

The crown is normally resilient or flexible such that it can be used for different wearer's and fit a variety of head shapes and sizes.

In addition, the position of the noise suppression portions along the length of the arm members can normally be adjusted for different wearers.

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to ear muffs which may at least partially overcome at least one of the abovementioned disadvantages or provide the consumer with a useful or commercial choice.

With the foregoing in view, the present invention in one form, resides broadly in ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on a carrier portion adapted to be supported on a wearer's head.

In an alternative form, the invention resides in a hardhat with removable ear muffs, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on the hard hat for removable attachment of the arm members thereto.

In an alternative form, the invention resides in eyeglasses with removable ear muffs, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on either side of the eyeglasses for removable attachment of the arm members thereto.

In an alternative form, the invention resides in ear muffs, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on a headband carrier portion for removable attachment of the arm members thereto.

In an alternative form, the invention resides in an occupational health and safety kit including ear muffs, a headband carrier portion, a hard hat and eyeglasses, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on each of the headband carrier portion, hard hat and eyeglasses for removable attachment of the arm members to each as required.

The earmuffs of the present invention are typically designed for attachment to various carrier or support assemblies to allow for use in different forms, with different limitations and different advantages.

According to alternative embodiments, the carrier or support assemblies may be a headband, a hardhat or eyeglasses.

The earmuffs of the present invention will typically be manufactured in light, medium and heavy duty configurations for different noise suppression levels and therefore different safety factors. Typically, different materials will be used for each.

The earmuffs will preferably have an ergonomic design with a quick and easy engagement and disengagement from the different carrier or support assemblies. Preferably, adjustment, attachment and removal will be accomplished without the use of tools or with only minimal use of tools.

Preferably, the earmuffs will be adjustable to suit their use, either before attachment, or once they are attached to the carrier or support assembly.

The earmuffs will typically include a pair of arms members, each arm member provided with a noise suppression portion mounted rotatably thereto at or adjacent to a first end of the arm.

Each arm will typically have the same shape. Each arm will preferably be rectilinear when viewed from the front of the arm. Each arm will typically have a plurality of shaped portions.

According to a particularly preferred embodiment, each arm will include a first portion for mounting the noise suppression portion thereon. Normally, the first portion will be arcuate.

There will preferably be an opening therein. Preferably, the opening will be a longitudinal opening. There will preferably be an enlarged portion at a first end thereof. The opening will preferably be a rectangular opening with a further rectangular enlarged portion at the first end. The enlarged portion of the opening will preferably allow attachment and removal of the noise suppression portion. The enlarged portion will also typically allow rotation of the noise suppression portion. The opening will typically be closed at the first end.

The longitudinal opening will preferably be defined by two elongate outer portions which stand proud of the surface of the first portion. There will further typically be a depression about the opening and extending parallel thereto. The depression will preferably be rectangular. Together with the outer portions, the depression will preferably frame the opening in order to form a track along the first portion of the arm. The track will typically define and limit the movement of the noise suppression portion along the arm.

The first portion will typically be positioned parallel to the wearer's head when in use.

Each arm will preferably also have an intermediate portion. The intermediate portion will typically be located between the first portion and an attachment portion of the arm. The intermediate portion will typically extend approximately perpendicularly to the first portion. The intermediate portion will typically extend from the first portion eight weeks approximately equal to the width of the noise suppression portion.

There will typically be an arcuate transition between the first portion and the intermediate portion of each arm.

Each arm will also typically include an attachment portion. The attachment portion will typically mount the first portion of the attachment system.

Each arm may include strengthening or rigidity increasing formations. In a preferred form, a strengthening rib may extend longitudinally over the length of the intermediate and attachment portions of each arm.

Each arm will typically have a noise suppression portion or muff. Typically, each muff will have a plastic or similar body, which is substantially dome shaped and hollow. Each noise suppression portion will preferably be provided with a padded periphery and/or interior in order that the noise suppression portion can be placed over a wearer's ear and the padded periphery substantially seals against the wearer's head.

There will typically be an attachment system provided in association with the outer portion of the dome shaped portion of each muff in order to attach the muff to the arm member.

In a particularly preferred embodiment, a threaded rod or similar will be provided extending outwardly from the rear of the dome shaped body. A rectangular bush and a threaded nut portion will also be provided. In use, the noise suppression portion will be located on the inside of a first portion of the arm with the rectangular bush located on the outer side of the arm in the rectangular depression. Engagement and tightening of the threaded nut portion clamps the rectangular bush in the depression and also clamps the arm between the bush and the rear of the noise suppression portion.

The noise suppression portion may have an additional guide portion adjacent to the attachment system. The guide portion will typically be configured with a rectangular bush mounted on either side of the arm in order to guide movement of the noise suppression portion along the track.

A first portion of an attachment system is located at a second end of each arm. The first portion of the attachment system is to allow attachment to a second portion of the attachment system which will be provided on a carrier portion adapted to be supported on a wearer's head.

A quick and easy attachment system is preferred. It is also preferred that the attachment system the operable to connect or disconnect the arms quickly and easily. For these reasons, a snap or click fit system is preferred where engagement portions are biased into an engaged condition and must be deformed to attach or detach the arms.

Two main embodiments of attachment system are preferred.

According to a first preferred embodiment, a male portion is provided with at least one and preferably a pair of deformable side portions, each with an engagement shoulder. The deformable side portions are typically located on either side of a central alignment tongue. The male portion typically engages with a female portion having a hollow body with at least one and preferably a pair of openings, one on each side of the body. In use, the body will typically receive the male portion therein such that the engagement shoulders of the male portion engage the surrounds of the openings. The side portions are deformed to pass the forward portion of the female portion and the resilience of the side portions maintains the engagement of the shoulders with the surrounds of the openings. Depression of the side portions will typically disengage the shoulders from the surrounds of the opening and allows separation of the male portion from the female portion.

According to this embodiment of the present invention, either the male or female portion can be the first portion of the attachment system, but typically the male portion is used in this capacity.

According to the second preferred embodiment, at least one and preferably a pair of attachment arms are provided, each arm having an L-shape with a fork portion of each arm extending transversely to the arm from which it extends. Again, deformation of the attachment arms is required in order to attach and detach the arm from the carrier portion. In use, each attachment arm will typically engage with a corresponding opening provided on the carrier portion. This embodiment may be simpler than the first embodiment but will typically not be as effective or as easy to operate as the first embodiment.

In use, the arms of the earmuffs will typically be attached to a carrier or support portion adapted to engage a wearer's head. There are three main embodiments preferred according to the present invention each provided with at least one second portion of the attachment system. The three main embodiments include a crown band, a hard hat and eyeglasses or goggles.

In a preferred form, the crown band will preferably be arcuate and adapted to overlie the crown of a person's head. The band will typically be padded with a removable padding 38 for comfort. Preferably, a second portion of the attachment system is provided at or adjacent each end of the band. It is also preferred that the band has substantial width in order to provide comfort.

In a preferred form, the hardhat may have any configuration. The earmuffs will typically be attached to the hardhat for use when ear and head protection are required but eye protection is not. The hardhat is preferably provided with one second portion of the attachment system on each lateral side of the body of the hardhat and may have a second portion located forwardly and/or rearwardly in order to mount additional elements relative to the hardhat. Normally, the second portions are located on the exterior rather than the interior of the hardhat. Typically, any second portions are integrally moulded with the exterior of the hardhat. When attached to the hardhat, each arm is approximately vertically oriented in the use condition.

Alternatively, the second portion may be attached to an integrally molded attachment portion provided on the hardhat. A hinged configuration is preferred here.

In a preferred form, the eyeglasses include at least one lens. Normally, a pair of lenses may be provided. Alternatively, a single unitary or monolithic lens may be provided which extend substantially across the wearer's face. The at least one lens will be appropriately mounted on the frame. This mounting may result in the at least one lens being mounted “to” the frame which normally occurs with the unitary lens or “in” the frame (within eyewires or rims) as normally occurs when a pair of lenses are provided.

The lenses can be any type of lens known-in the field. In particular, the lenses may be polarised, tinted, photochromic, UV resistant and/or UV protective, prescription, or provide impact protection. They may be coloured or clear.

The frame of the eyewear of the present invention is typically at least partially rigid. Frames for conventional glasses and the like normally include at least some of the following components:
Frame front: Front part of the eyeglass frame that holds the lenses in place and bridges the top of the nose.
Eye wires (rims): Part of the frame front into which the lenses are inserted.
Bridge: The area between the lenses that extends over the nose and supports the majority of the weight of the eyeglasses. Examples include:
   A keyhole bridge is shaped like an old-fashioned keyhole and rests on the sides of the upper part of the nose. This style is for those with small or flat nose bridges.
   A saddle bridge is shaped like a saddle and spreads the weight of the frame across the sides and the top of the nose. This style works well for heavy glasses or for those who are sensitive to pressure.
   An adjustable bridge includes nose pads that can be adjusted for fit and comfort.
   A double bridge has a reinforcing bar over the top of the bridge.

The frame of the present invention will typically be at least partially rigid. Normally, at least the frame front will be rigid.

It is preferred that the eyeglasses/goggles of the invention do not include ear stems and instead are maintained relative to a user's face by either a resilient strap attached to the frame, or through attachment of the frame to ear muffs.

The eyeglasses will be provided with one second portion of the attachment system on each lateral side of the frame. Normally, the arms of the ear muffs are attached to the frame of the eyeglasses. In this configuration, the arms will extend substantially horizontally in use.

In this configuration, the noise suppression portions are rotated relative to the arms in order to correctly cover a wearer's ears. This configuration is typically used when hardhat protection is not required but hearing and eye protection is.

The second portion provided on each lateral side of the frame of the eyeglasses may be used in the alternative to attach a strap provided with first portions of the attachment system.

It is preferred that the carrier or support portions are sold together with the arm members and attached noise suppression portions as a kit together with the strap for the eyeglasses.

### BRIEF DESCRIPTION OF THE DRAWINGS

Various embodiments of the invention will be described with reference to the following drawings, in which:

FIG. 1 is a front view of a set of ear muffs according to a preferred embodiment of the present invention.

FIG. 2 is a view of the component portions of the ear muffs illustrated in FIG. 1.

FIG. 3 shows three alternate views of the noise suppression portion of the ear muffs illustrated in FIG. 1.

FIG. 4 is an isometric view of a hard hat of a preferred embodiment with ear muffs attached.

FIG. 5 illustrates an ear muff attachment for the hard hat illustrated in FIG. 4.

FIG. 6 is a side view of the hard hat and ear muffs illustrated in FIG. 4.

FIG. 7 is a side view of ear muff attachment for the hard hat illustrated in FIG. 5.

FIG. 8 is a side view of a set of eyeglasses/goggles with ear muffs attached.

FIG. 9 is a side view of an ear muff attachment for the eyeglasses/goggles illustrated in FIG. 8.

FIG. 10 is an isometric view of a set of eyeglasses/goggles with ear muffs attached.

FIG. 11 is an alternative view of the ear muff attachment for the eyeglasses/goggles illustrated in FIG. 8.

FIG. 12 is a side view of the eyeglasses/goggles illustrated in FIG. 8 with an attachment strap in place of the ear muffs.

FIG. 13 is a side view of the eyeglasses/goggles without strap or ear muffs.

FIG. 14 is an isometric view of a hard hat of a preferred embodiment with ear muffs attached.

FIG. 15 illustrates a hard hat as illustrated in FIG. 14 without any attachments.

FIG. 16 is a front view of a hard hat as illustrated in FIG. 15.

FIG. 17 is a side view of the hard hat and ear muffs illustrated in FIG. 14.

FIG. 18 is illustrates a hard hat as illustrated in FIG. 14 with a front lighting attachment.

FIG. 19 illustrates a preferred form of lighting attachment and tamp.

FIG. 20 illustrates a preferred form of lighting attachment.

FIG. 21 is a top view of a hard hat according to a preferred 15 embodiment.

FIG. 22 illustrates a preferred form of lighting attachment from a different angle.

FIG. 23 illustrates a preferred embodiment of headband according to a second embodiment of the present invention.

FIG. 24 is a front view of a set of ear muffs according to a second preferred embodiment of the present invention.

FIG. 25 illustrates an ear muff attachment according to a second embodiment configured to be used with eyeglasses/goggles.

FIG. 26 illustrates an ear muff attachment illustrated in FIG. 24 25 configured to be used with a hard hat or headband.

FIG. 27 illustrates an ear muff attachment according to a second embodiment configured to be used with eyeglasses/goggles.

FIG. 28 illustrates eyeglasses/goggles of a second embodiment without strap or ear muffs.

FIG. 29 illustrated the eyeglasses/goggles illustrated in FIG. 28 with ear muffs attached.

FIG. 30 is a side view of the eyeglasses/goggles illustrated in FIG. 28 with an attachment strap in place of the ear muffs.

FIG. 31 illustrates an ear muff attachment illustrated in FIG. 24 configured to be used with a hard hat or headband.

FIG. 32 is a side view of the hard hat and ear muffs of a second preferred embodiment.

FIG. 33 is a side view of a noise suppression portion of ear muffs according to another preferred embodiment of the present invention.

FIG. 34 is a perspective view of the ear muffs illustrated in FIG. 33.

FIG. 35 illustrates a preferred embodiment of headband according to a preferred embodiment of the ear muffs illustrated in FIG. 33.

FIG. 36 is a front view of a set of ear muffs according to the preferred embodiment of the ear muffs illustrated in FIG. 33.

FIG. 37 illustrates an ear muff attachment illustrated in FIG. 35 36 configured to be used with eyeglasses/goggles.

FIG. 38 illustrates an ear muff attachment illustrated in FIG. 35 36 configured to be used with a hard hat or headband.

FIG. 39 is a front view of a set of ear muffs according to another preferred embodiment of the ear muffs illustrated in FIG. 33.

FIG. 40 is a view of the component portions of the ear muffs illustrated in FIG. 39.

FIG. 41 shows three alternate views of the noise suppression portion of the ear muffs illustrated in FIG. 33.

FIG. 42 is a front view of a set of ear muffs according to yet another preferred embodiment of the present invention.

FIG. 43 is a view of the component portions of the ear muffs illustrated in FIG. 42.

FIG. 44 shows three alternate views of the noise suppression portion of the ear muffs illustrated in FIG. 42.

FIG. 45 is a view of the component portions of the ear muffs according to another preferred embodiment.

FIG. 46 is a perspective view of the ear muffs illustrated in FIG. 45.

FIG. 47 is a side view of the ear muffs illustrated in FIG. 45.

FIG. 48 is a front top left side perspective view of an ear muff attachment according to another preferred embodiment of the present invention.

FIG. 49 is a front side view of the ear muff attachment illustrated in FIG. 48.

FIG. 50 is a rear top right side perspective view of the ear muff attachment illustrated in FIG. 48.

FIG. 51 is a right side view of the ear muff attachment illustrated in FIG. 48.

FIG. 52 is a front view of the ear muff attachment illustrated in FIG. 48.

FIG. 53 is a rear view of the ear muff attachment illustrated in FIG. 48.

FIG. 54 is a top view of the ear muff attachment illustrated in FIG. 48.

FIG. 55 is a bottom view of the ear muff attachment illustrated in FIG. 48.

FIG. 56 is a front top left side perspective view of an ear muff attachment according to another preferred embodiment of the present invention.

FIG. 57 is a rear top right side perspective view of the ear muff attachment illustrated in FIG. 56.

FIG. 58 is a front side view of the ear muff attachment illustrated in FIG. 56.

FIG. 59 is a right side view of the ear muff attachment illustrated in FIG. 56.

FIG. 60 is a front view of the ear muff attachment illustrated in FIG. 56.

FIG. 61 is a rear view of the ear muff attachment illustrated in FIG. 56.

FIG. 62 is a top view of the ear muff attachment illustrated in FIG. 56.

FIG. 63 is a bottom view of the ear muff attachment illustrated in FIG. 56.

### DETAILED DESCRIPTION OF THE PREFERRED EMBODIMENT

According to the preferred embodiment, a set of ear muffs which can be configured for different uses and attachments is provided.

A pair of embodiments of ear muffs is illustrated. The ear muffs of both embodiments include the same overall features with the differences between the embodiments being in the type of attachment system used.

In the illustrated embodiments, the ear muffs 10 are provided as a set or pair including a pair of arm members 11, each arm member 11 provided with a noise suppression portion 12 mounted rotatably thereto adjacent a first end of each arm member 11 and having a first portion 13 of an attachment system located at a second opposite end of each arm member 11, the first portion 13 of the attachment system attachable to a second portion 14 of the attachment system provided on a carrier portion adapted to be supported on a wearer's head.

The earmuffs of the present invention are typically designed for attachment to various carrier or support assemblies to allow for use in different forms, with different limitations and different advantages.

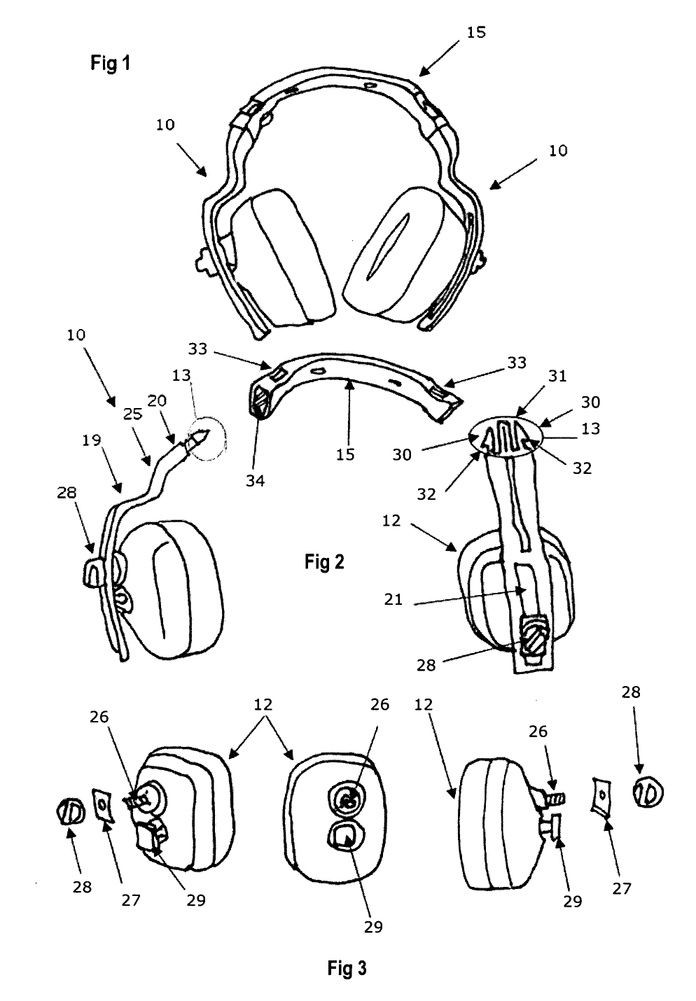

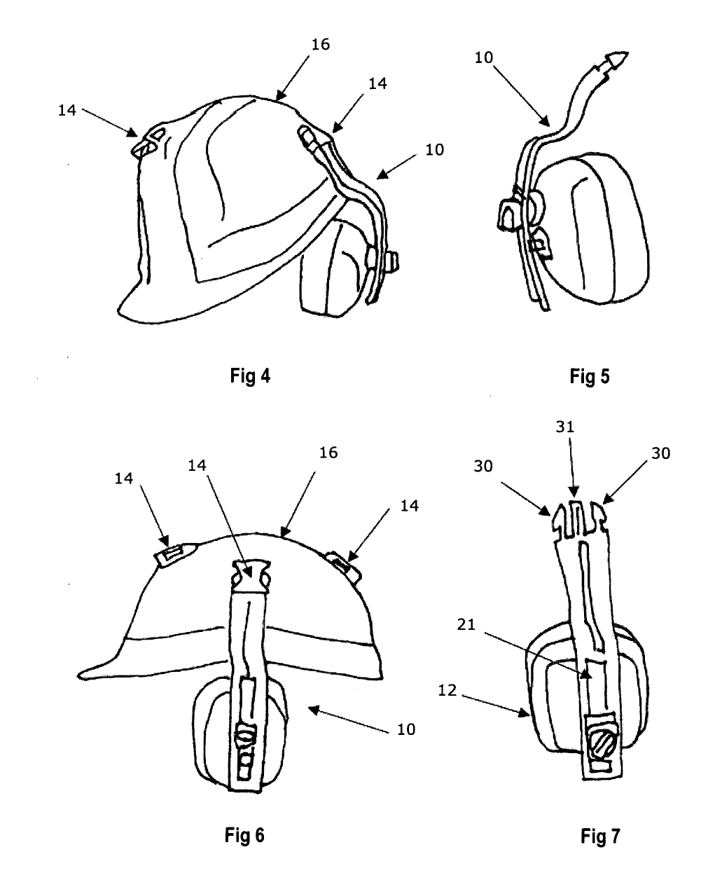

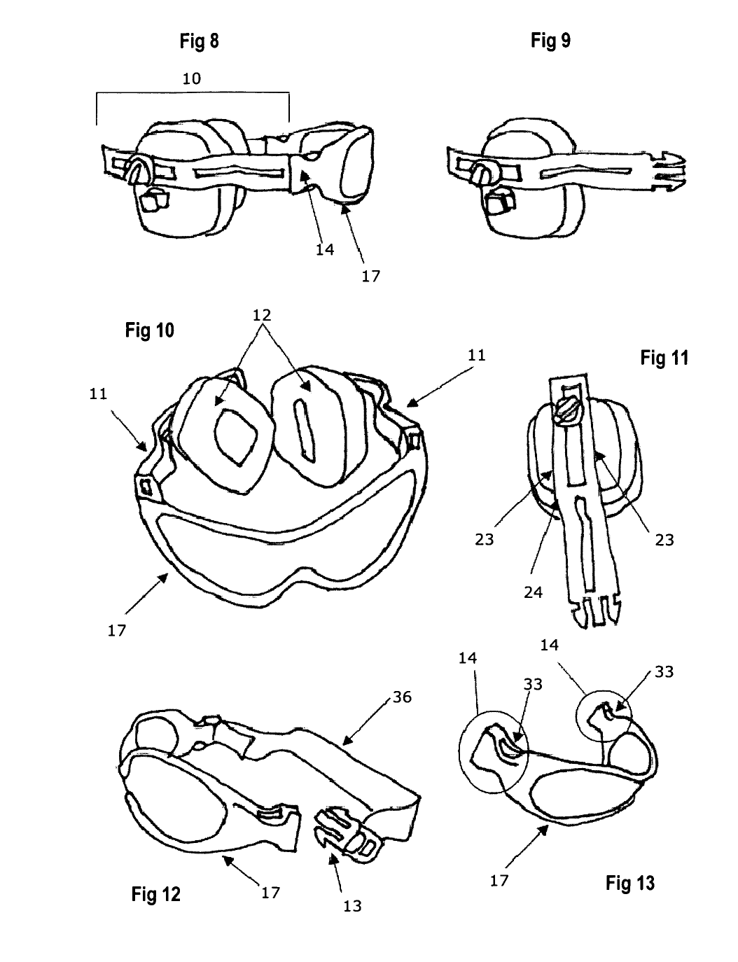

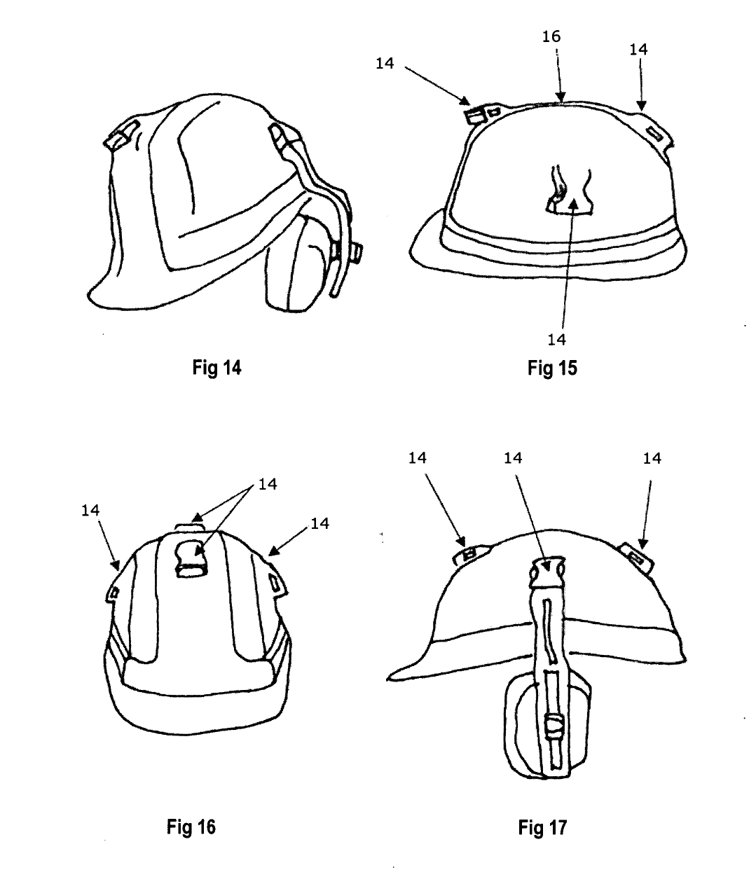

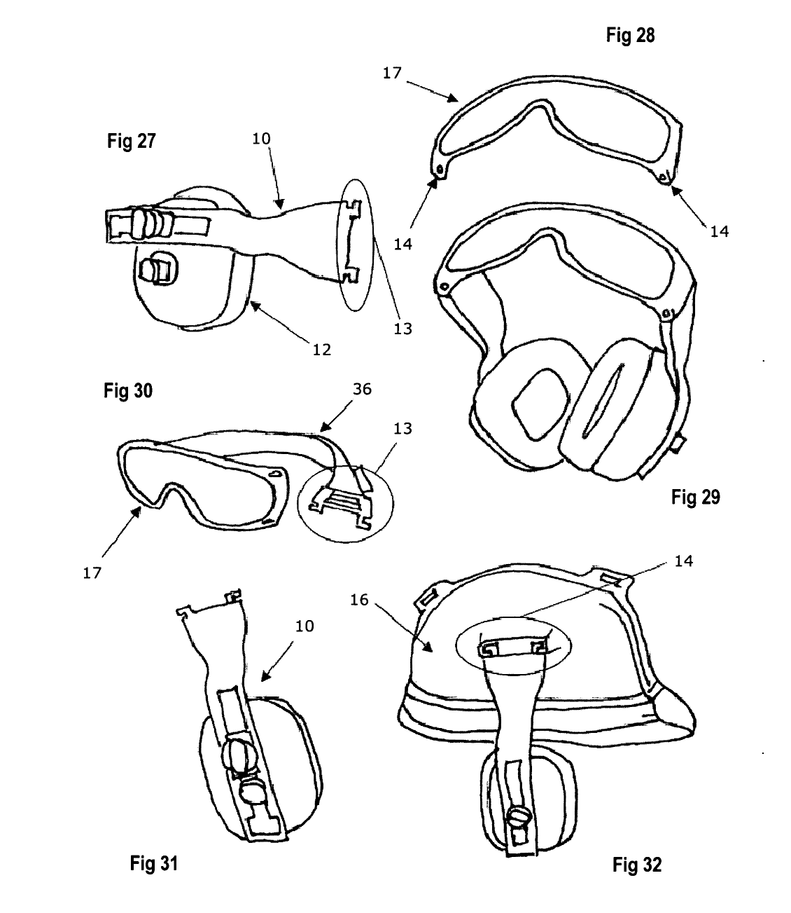

According to alternative embodiments, the carrier or support assemblies may be a headband 15 as illustrated in FIGS. 1, 2, 23 and 24, a hardhat 16 as illustrated in FIGS. 4, 6, 14 to 17, and 32, or eyeglasses 17 as illustrated in FIGS. 8 to 13 and 28 to 30.

The attachment system will preferably have a quick and easy engagement and disengagement from the different carrier or support assemblies. Adjustment, attachment and removal will be accomplished without the use of tools or with only minimal use of tools.

Each arm 11 has the same shape with the arms of the pair being mirror images of one another. Each arm 11 illustrated is rectilinear when viewed in plan having a plurality of shaped portions.

According to the illustrated embodiment, each arm 11 includes a first portion 18 for mounting the noise suppression portion 12 thereon. Normally, the first portion is arcuate.

There is a longitudinal opening 21 through the first portion 18 having an enlarged portion 22 at a first end thereof. The enlarged portion 22 of the opening 21 allows attachment and removal of the noise suppression portion 12. The enlarged portion 22 also allows rotation of the noise suppression portion 12.

According to the preferred embodiment illustrated in FIG. 11, the longitudinal opening 21 is defined by two elongate outer portions 23 which stand proud of the surface of the first portion 18. There is a rectangular depression 24 about the opening 21 and extending parallel thereto. Together the outer portions 23, and the depression 24 frame the opening 21 in order to form a track along the first portion 18 of the arm 11 to define and limit the movement of the noise suppression portion 12 along the arm 11.

Each arm 11 also has an intermediate portion 19. The intermediate portion 19 is located between the first portion 18 and an attachment portion 20 of the arm 11. The intermediate portion 19 extends approximately perpendicularly to the first portion 18. The intermediate portion 19 extends from the first portion 18 a length approximately equal to the width of the noise suppression portion 12.

There will typically be an arcuate transition between the first portion 18 and the intermediate portion 19 of each arm 11.

Each arm 11 also includes an attachment portion 20. The attachment portion 20 mounts the first portion 13 of the attachment system.

In the illustrated form, a strengthening rib 25 extends longitudinally over the length of the intermediate 19 and attachment portions 21 of each arm.

Each arm 11 mounts a noise suppression portion 12 or muff. Typically, each muff 12 has a plastic or similar body which is substantially dome shaped and hollow. Each noise suppression portion 12 has provided with a padded periphery and/or interior in order that the noise suppression portion 12 can be placed over a wearer's ear and the padded periphery seals against the wearer's head.

There is an attachment system provided in association with the outer portion of the dome shaped portion of each muff in order to attach the muff 12 to the arm 11.

In the embodiment illustrated in FIG. 3, a threaded rod 26 extends outwardly from the rear of the dome shaped body. A rectangular bush 27 and a threaded nut portion 28 are also provided. In use, the noise suppression portion 12 will be located on the inside of a first portion 18 of the arm 11 with the rectangular bush 27 located on the outer side of the arm 11 in the rectangular depression 24.

Engagement and tightening of the threaded nut 28 clamps the rectangular bush 27 in the depression 24 and also clamps the arm 11 between the bush 27 and the rear of the noise suppression portion 12.

The noise suppression portion illustrated in FIG. 3 has an additional 10 guide portion 29 adjacent to the attachment system. The guide portion 29 has a rectangular bush mounted on either side of the arm 11 in order to guide movement of the noise suppression portion 12 along the track.

A first portion 13 of an attachment system is located at a second end of each arm 11. The first portion 13 of the attachment system is to allow attachment to a second portion 14 of the attachment system which is provided on the carrier portion adapted to be supported on a wearer's head.

Two main embodiments of attachment system are illustrated.

According to a first preferred embodiment illustrated in FIGS. 1 to 22, a male portion is provided with a pair of deformable side portions 30, each with an engagement shoulder 32. The deformable side portions 30 are located on either side of a central alignment tongue 31. The male portion typically engages with a female portion having a hollow body 34 with a pair of openings 33, one on each side of the body 34. In use, the body 34 receives the male portion therein such that the engaged in shoulders 32 of the male portion engage the surrounds of the openings 33. The side portions 30 are deformed to pass the forward portion of the female portion and the resilience of the side portions 30 maintains the engagement of the shoulders 32 with the surrounds of the openings 33. Depression of the side portions 30 will disengage the shoulders 32 from the surrounds of the opening 33 and allows separation of the male portion from the female portion.

According to this embodiment of the present invention, either the male or female portion can be the first portion of the attachment system, but typically the male portion is used in this capacity.

According to the second preferred embodiment illustrated in FIGS. 23 to 32, a pair of attachment arms 35 is provided, each arm having an L-shape with a first portion of each arm extending transversely to the arm from which it extends. Again, deformation of the attachment arms 35 is required in order to attach and detach the arm from the carrier portion. In use, each attachment arms 35 will engage with a corresponding opening 36 provided on the carrier portion. This embodiment is simpler than the first embodiment but will typically not be as effective or is easy to operate as the first embodiment.

In use, the arms of the earmuffs 10 will typically be attached to a carrier or support portion adapted to engage a wearer's head. There are three main embodiments illustrated, each provided with a second portion of the attachment system. The three main embodiments include a crown or head band, a hard hat and eyeglasses or goggles.

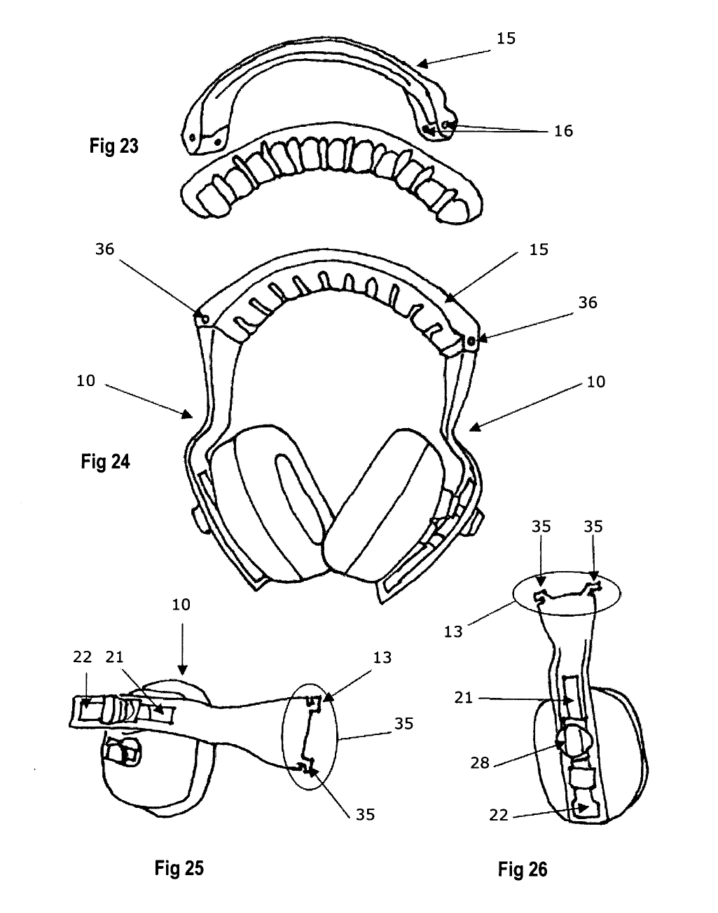

In the forms as illustrated in FIGS. 1, 2, 23 and 24, the head band 15 is arcuate and adapted to overlie the crown of a person's head. The head band can be padded for comfort as illustrated in FIGS. 23 and 24. A second portion of the attachment system is provided at or adjacent each end of the head band 15.

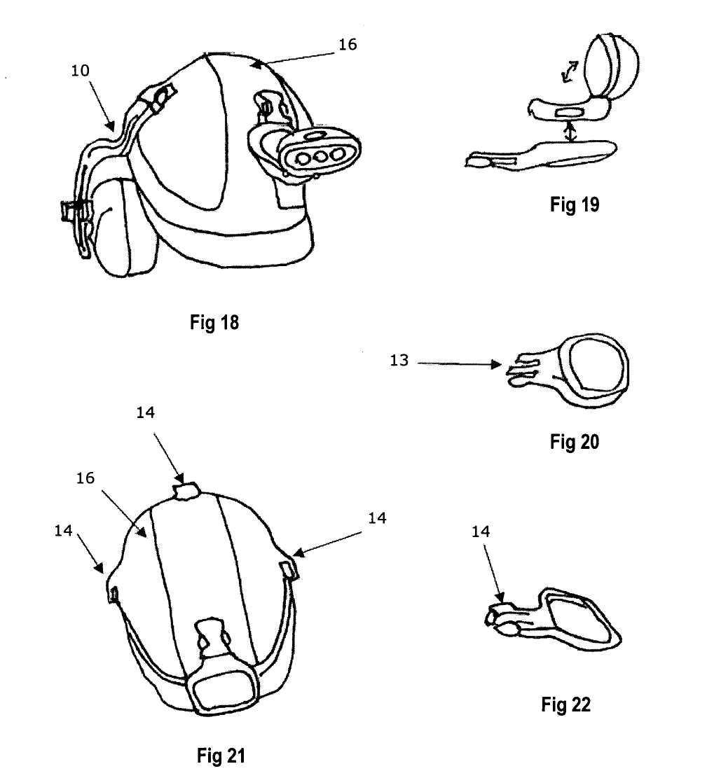

In the forms as illustrated in FIGS. 4, 6, 14 to 17, and 32, the earmuffs 10 are attached to the hardhat 16 for use when ear and head protection are required but eye protection is not. The hardhat 16 is provided with one second portion 14 of the attachment system on each lateral side of the body 35 of the hardhat 16 and also has a second portion 14 located forwardly and/or rearwardly in order to mount additional elements relative to the hardhat 16 such as the lighting attachment illustrated in FIGS. 18 to 22. The second portions 14 are located on the exterior rather than the interior of the hardhat and are normally moulded with the exterior of the hardhat. When attached to the hardhat 16, each arm 11 is approximately vertically oriented in the use condition, as illustrated in FIG. 6 for example.

In the forms as illustrated in FIGS. 8 to 13 and 28 to 30, the eyeglasses/goggles 17 include at least one lens which extends substantially across the wearer's face. The lens will be appropriately mounted on the frame. This mounting may result in the at least one lens being mounted “to” the frame which normally occurs with the unitary lens or “in” the frame (within eyewires or rims) as normally occurs when a pair of lenses are provided.

The lenses can be any type of lens known-in the field. In particular, the lenses may be polarised, tinted, photochromic, UV resistant and/or UV protective, prescription, or provide impact protection. They may be coloured or clear.

It is preferred that the eyeglasses/goggles 17 of the invention do not include ear stems and instead are maintained relative to a user's face by either a resilient strap 36 attached to the frame, or through attachment of the frame to ear muffs 10.

The eyeglasses/goggles are provided with one second portion 14 of the attachment system on each lateral side of the frame. Normally, the arms 11 of the ear muffs 10 are attached to the frame of the eyeglasses/goggles 17. In this configuration, the arms 1 will extend substantially horizontally in use as illustrated in FIG. 8 for example.

In this configuration, the noise suppression portions 12 are rotated relative to the arms 11 in order to correctly cover a wearer's ears. This configuration is typically used when hardhat protection is not required but hearing and eye protection is.

The second portion 14 provided on each lateral side of the frame of the eyeglasses/goggles 17 can used in the alternative to attach a strap 36 provided with first portions 13 of the attachment system.

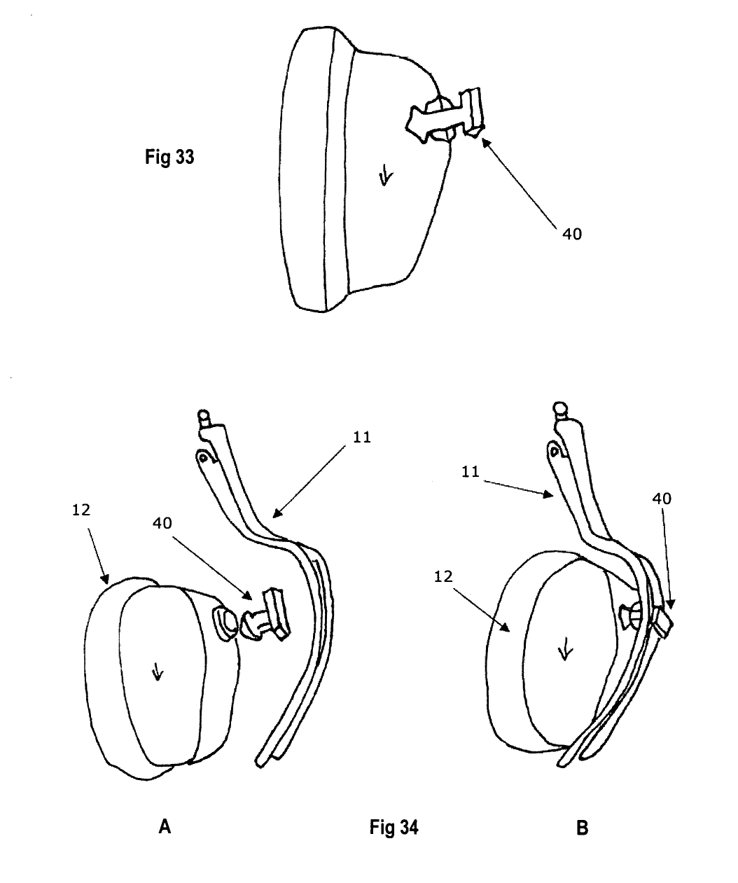

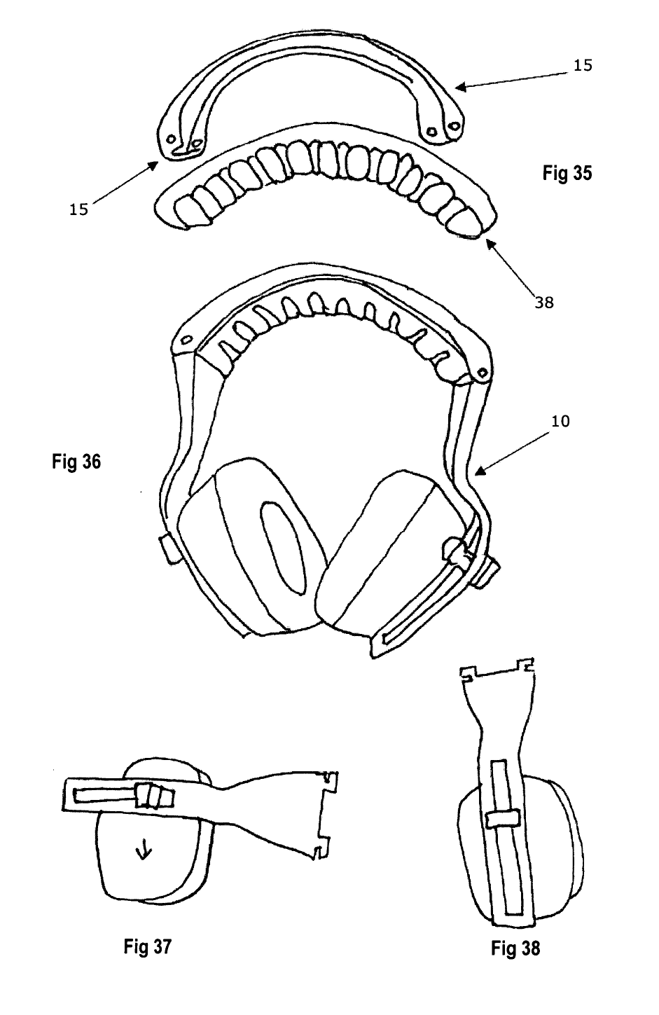

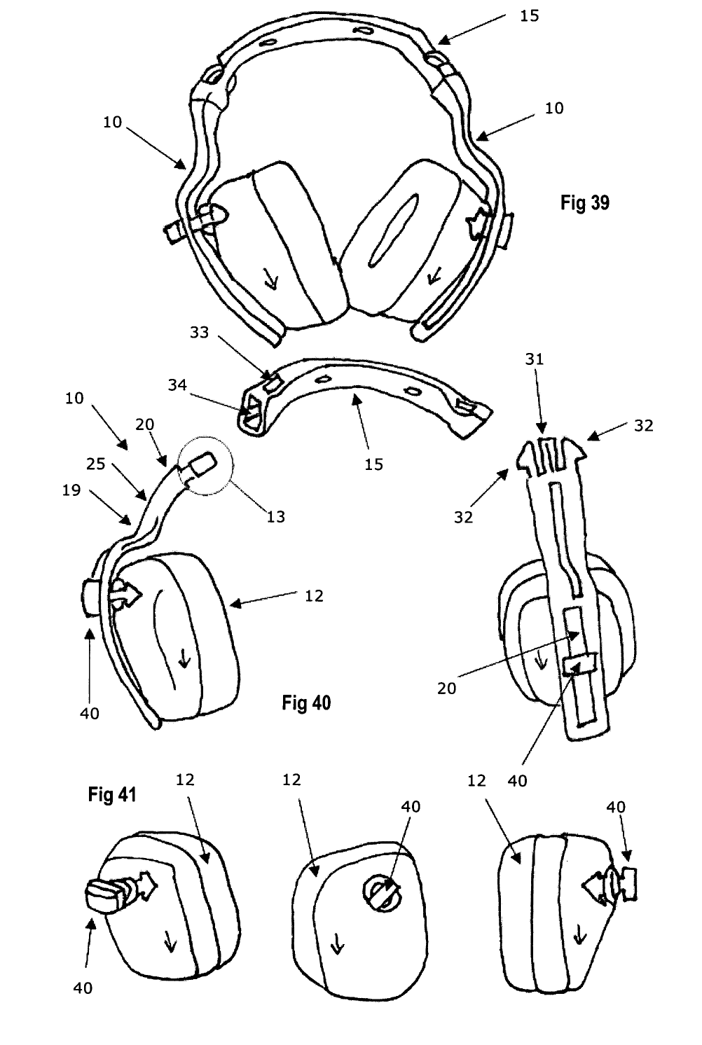

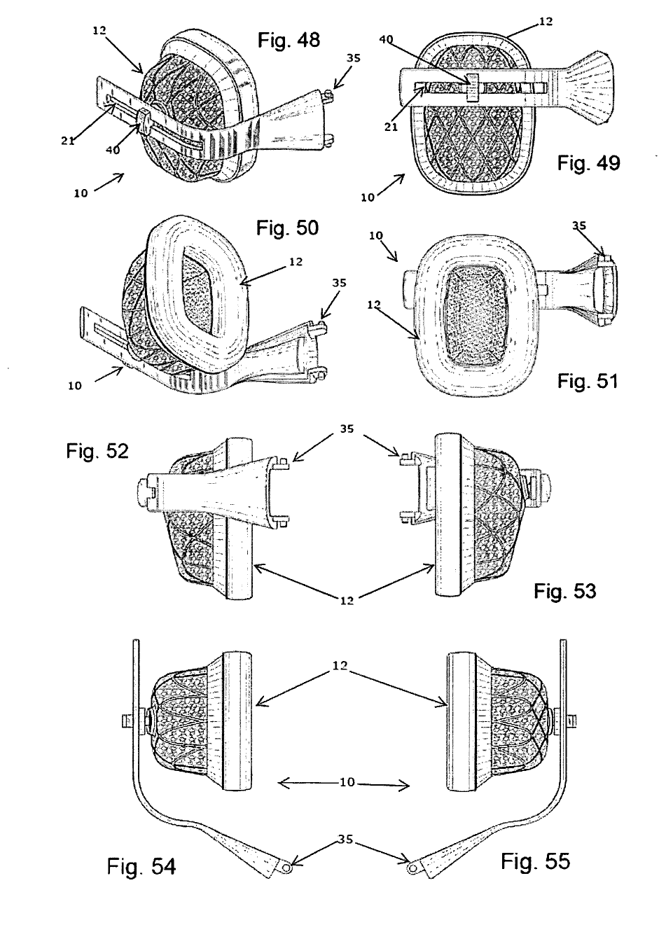

Reference is now made to FIGS. 33-41, and 48-55, which show another preferred embodiment of the present invention. The attachment system 20 of this embodiment comprises a locking knob 40 removably attached to the noise suppression portion 12 through a joining means. The locking knob 40 has two configurations: the locking position, as shown in FIG. 34B for securing the noise suppression portion 12 to the arm member 11, and the unlocking position, as shown in FIG. 34A, which allows the noise suppression portion to be removed from the arm member.

As shown in FIG. 34A, the locking knob 40 has a unlocking position. In this configuration, the head of the locking knob is in a position which can pass through the longitudinal opening 21 of the arm member 11 for engaging or disengaging the arm member. When the noise suppression portion 12 engages the arm member 11 in the unlocking position, it can be turned into the locking position, as shown in FIG. 34B, such that the spatial configuration of the locking knob can prevent the noise suppression portion from disengaging the arm member. Preferably, a clamping means is provided to hold the noise suppression portion 12 onto the arm member 11.

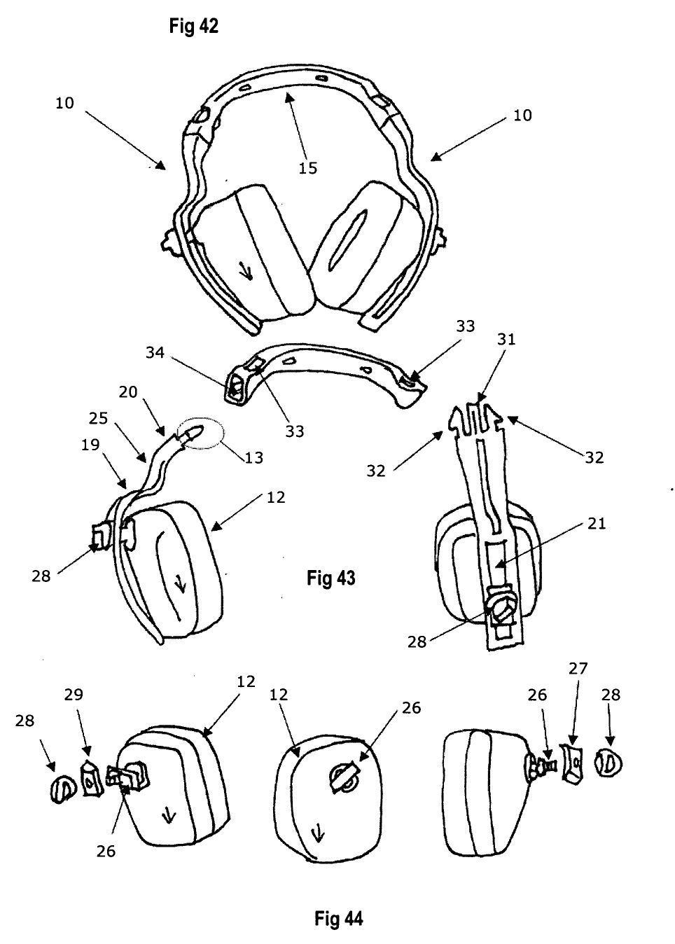

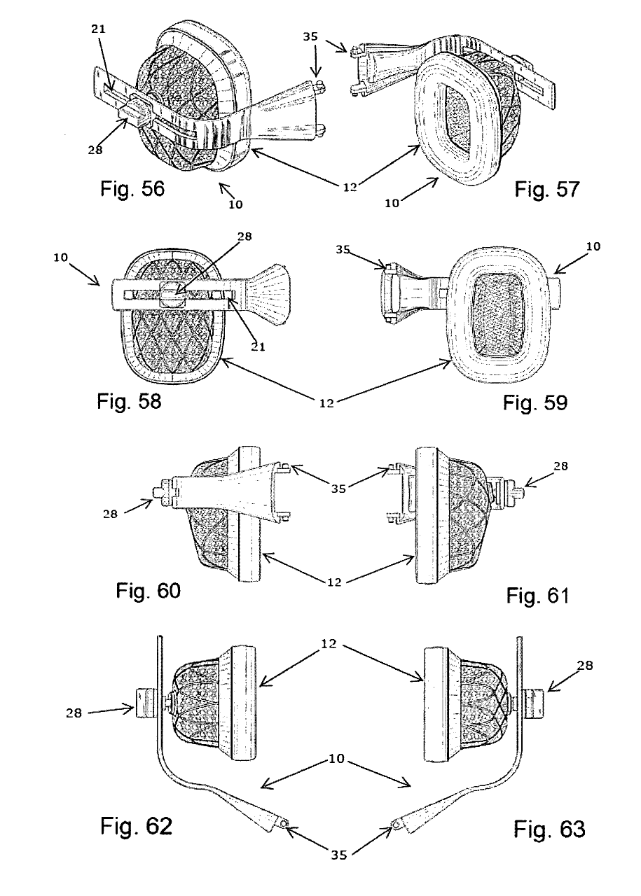

Reference is now made to FIGS. 32-47, and 56-63, which show yet another preferred embodiment of the present invention. The attachment system 20 of this embodiment comprises a locking means that is removably attached to the noise suppression portion 12 through a joining means. The locking means comprises a thread rod portion 26, a bush 27 and a nut 28.

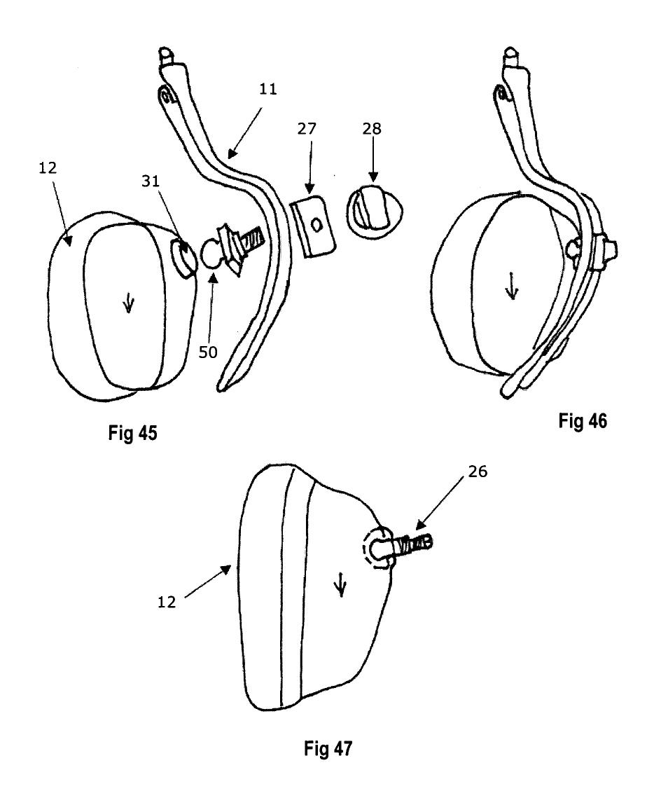

As shown in FIG. 45, when in use, the noise suppression portion 12 with the thread rod portion 26 attached thereon will be located on the inside of the arm member 11 and will have the thread rod extending outwardly. The rectangular bush 27 and the nut 28 are located on the outer side of the arm member 11 for mounting the arm member onto the thread rod portion thereby securing the noise suppression portion 12 to the arm member 11.

Preferably, as shown in FIG. 45, the joining means comprises a ball portion 50 and socket portion 51 which allows the noise suppression portion 12 freely rotatable around a plane perpendicular to the vertical axis of the joining member.

In the present specification and claims (if any), the word “comprising” and its derivatives including “comprises” and “comprise” include each of the stated integers but does not exclude the inclusion of one or more further integers.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. Ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on a carrier portion adapted to be supported on a wearer's head.

2. Ear muffs as claimed in claim 1 wherein each arm member has a longitudinal opening therein to allow adjustment of the position of the noise suppression portion.

3. Ear muffs as claimed in claim 2 wherein the longitudinal opening is defined by two elongate outer portions which stand proud of the surface of the arm member and parallel to the length of the opening forming a guide depression in the arm member about the opening and extending parallel thereto.

4. Ear muffs as claimed in claim 3 wherein a threaded rod is provided extending outwardly from the rear of the noise suppression portion, associated with a rectangular bush and a threaded nut portion such that in use, the noise suppression portion is located on the inside of the arm member with the rectangular bush located on the outer side of the arm in the rectangular depression, the threaded nut portion clamping the rectangular bush in the depression and the arm member between the bush and a rear portion of the noise suppression portion.

5. Ear muffs as claimed in claim 3 wherein the noise suppression portion has a guide portion to guide movement of the noise suppression portion along the arm member.

6. Ear muffs as claimed in claim 1 wherein the attachment system includes a male portion provided with at least one deformable side portion, each with an engagement shoulder engageable with a female portion having a hollow body with at least one opening, one on each side of the body such that in use, the body receives the male portion therein, temporarily deforming the at least one deformable side portion such that the engagement shoulder of the male portion engages the surrounds of the openings.

7. Ear muffs as claimed in claim 6 wherein the carrier portion is chosen from the group of a headband, a hardhat or eyeglasses/goggles and wherein the male portion of the attachment system is provided on the arm member and at least one female portion of the attachment system is provided on the headband, hardhat or eyeglasses/goggles.

8. Ear muffs as claimed in any one of claim 1 wherein the attachment system includes at least one attachment arm, each arm having an L-shaped portion with a portion extending transversely to the arm from which it extends, each L-shaped portion engageable in use with a corresponding opening provided on the carrier portion.

9. Ear muffs as claimed in claim 8 wherein the carrier portion is chosen from the group of a headband, a hardhat or eyeglasses/goggles and wherein the at least one attachment arm is provided on the arm member and at least one opening is provided on the headband, hardhat or eyeglasses/goggles.

10. A hardhat with removable ear muffs, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on the hard hat for removable attachment of the arm members thereto.

11. A hardhat with removable ear muffs as claimed in claim 10 wherein the hardhat is provided with one second portion of the attachment system on each lateral side of the hardhat.

12. A hardhat with removable ear muffs as claimed in claim 10 wherein a second portion is located forwardly and/or rearwardly in order to mount additional elements relative to the hardhat.

13. A hardhat with removable ear muffs as claimed in any one of claim 10 wherein the second portions are integrally moulded with the exterior of the hardhat.

14. Eyeglasses with removable ear muffs, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on either side of the eyeglasses for removable attachment of the arm members thereto.

15. Eyeglasses with removable ear muffs as claimed in claim 14 wherein the eyeglasses include a frame with one second portion of the attachment system on each lateral side of the frame to attach the arm members thereto and the eyeglasses are maintained relative to a user's face via the ear muffs.

16. Eyeglasses with removable ear muffs as claimed in claim 14 or 15 wherein the noise suppression portions are rotated relative to the arms in order to correctly cover a wearer's ears.

17. Ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on a headband carrier portion for removable attachment of the arm members thereto.

18. Ear muffs as claimed in claim 17 wherein the headband carrier portion is arcuate and, adapted to overlie the crown of a person's head with a second portion of the attachment system provided at or adjacent each end of the band.

19. An occupational health and safety kit including ear muffs, a headband carrier portion, a hard hat and eyeglasses, the ear muffs including a pair of arm members, each arm member provided with a noise suppression portion mounted rotatably thereto adjacent a first end of each arm member and having a first portion of an attachment system located at a second opposite end of each arm member, the first portion of the attachment system attachable to a second portion of the attachment system provided on each of the headband carrier portion, hard hat and eyeglasses for removable attachment of the arm members to each as required.
